(19) United States
(12) Patent Application Publication
(10) Pub. No.: US 2011/0251219 A1
(43) Pub. Date: Oct. 13, 2011

(54) TREATMENT FOR GLOMERULONEPHRITIS WITH 2-[4-(7-ETHYL-5H-PYRROLO[2,3-B]PYRAZIN-6-YL)PHENYL]PROPAN-2-OL

(75) Inventors: Thomas OLIGINO, Bridgewater, NJ (US); Timothy A. GILLESPY, Bridgewater, NJ (US); Ramalinga DHARANIPRAGADA, Bridgewater, NJ (US); Elizabeth M. ALLEN, Bridgewater, NJ (US)

(73) Assignee: SANOFI, Paris (FR)

(21) Appl. No.: 13/151,916

(22) Filed: Jun. 2, 2011

Related U.S. Application Data
(63) Continuation of application No. PCT/US2009/066304, filed on Dec. 2, 2009.
(60) Provisional application No. 61/119,476, filed on Dec. 3, 2008.

(51) Int. Cl. A61K 31/519; A61P 13/12

(52) U.S. Cl. 514/265.1

(57) ABSTRACT

A treatment for glomerulonephritis, and other renal dysfunctions using a compound of Formula I is disclosed.

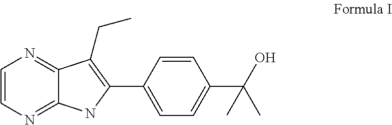

This application is a continuation of International Application No. PCT/US2009/066304, filed Dec. 2, 2009, which claims the benefit of priority of U.S. Provisional Application No. 61/119,476, filed Dec. 3, 2008, both of which are incorporated herein by reference.

### FIELD OF THE INVENTION

This invention is directed to a method of therapy for human and non-human patients suffering from, or subject to, glomerulonephritis.

### BACKGROUND OF THE INVENTION

Glomerulonephritis (GN) is a general name for disorders involving inflammation of the renal glomeruli. GN can be classified by the type of glomerular injuries involved, including antibody deposition, complement activation, cellular proliferation, and glomerulosclerosis. These structural and functional abnormalities usually lead to hematuria, proteinurea, renal hypertension, and renal insufficiency.

Current treatments for glomerulonephritis are focused on supportive therapies, in conjunction frequently with nonspecific immunosuppressive drugs, or with an antibody treatment sold under the name Lucentis®. Depending on the type of glomerulonephritis, the prognosis for patients is currently poor. Hricik, Chung-Park and Sedor. Glomerulonephritis. N Engl J Med 1998; 339:888-899. There is a clear need for effective therapies for this disorder, especially for effective oral therapies for this disorder.

In view of the current situation regarding therapies for treating glomerulonephritis, it is clear that there is a need for more effective and better tolerated therapies.

Protein kinases participate in the signalling events which control the activation, growth and differentiation of cells in response to extracellular mediators and to changes in the environment. In general, these kinases fall into several groups; those which preferentially phosphorylate serine and/or threonine residues and those which preferentially phosphorylate tyrosine residues [S. K. Hanks and T. Hunter, FASEB. J., 1995, 9, pages 576-596]. The serine/threonine kinases include for example, protein kinase C isoforms [A. C. Newton, J. Biol. Chem., 1995, 270, pages 28495-28498] and a group of cyclin-dependent kinases such as cdc2 [J. Pines, Trends in Biochemical Sciences, 1995, 18, pages 195-197]. The tyrosine kinases include membrane-spanning growth factor receptors such as the epidermal growth factor receptor [S. Iwashita and M. Kobayashi, Cellular Signalling, 1992, 4, pages 123-132], and cytosolic non-receptor kinases such as p56tck, p59fYn, ZAP-70 and csk kinases [C. Chan et. al., Ann. Rev. Immunol., 1994, 12, pages 555-592].

Inappropriately high protein kinase activity has been implicated in many diseases resulting from abnormal cellular function. This might arise either directly or indirectly, for example by failure of the proper control mechanisms for the kinase, related for example to mutation, over-expression or inappropriate activation of the enzyme; or by over- or underproduction of cytokines or growth factors also participating in the transduction of signals upstream or downstream of the kinase. In all of these instances, selective inhibition of the action of the kinase might be expected to have a beneficial effect.

Syk is a 72-kDa cytoplasmic protein tyrosine kinase that is expressed in a variety of hematopoietic cells and is an essential element in several cascades that couple antigen receptors to cellular responses. Thus, Syk plays a pivotal role in signalling of the high affinity IgE receptor, FcεR1, in mast cells and in receptor antigen signalling in T and B lymphocytes. The signal transduction pathways present in mast, T and B cells have common features. The ligand binding domain of the receptor lacks intrinsic tyrosine kinase activity. However, they interact with transducing subunits that contain immunoreceptor tyrosine based activation motifs (ITAMs) [M. Reth, Nature, 1989, 338, pages 383-384]. These motifs are present in both the β and γ subunits of the FcεR1, in the ζ-subunit the of T cell receptor (TCR) and in the IgGα and IgGβ subunits of the B cell receptor (BCR). [N. S. van Oers and A. Weiss, Seminars in Immunology, 1995, 7, pages 227-236] Upon binding of antigen and multimerization, the ITAM residues are phosphorylated by protein tyrosine kinases of the Src family. Syk belongs to a unique class of tyrosine kinases that have two tandem Src homology 2 (SH2) domains and a C terminal catalytic domain. These SH2 domains bind with high affinity to ITAMs and this SH2-mediated association of Syk with an activated receptor stimulates Syk kinase activity and localises Syk to the plasma membrane.

The present invention provides methods for treating (i.e., delaying the onset of, slowing the progression of, and/or reversing) kidney disorders (e.g., renal glomerulonephritis, and/or renal fibrosis, and/or diabetic nephropathy). Certain of these methods involve administering a Syk kinase inhibitor as a pharmaceutically effective amount, to a patient in need thereof.

The kidneys are a major target organ of hypertension. Prolonged hypertension induces various renal impairments, mainly through renovascular lesions. Among them, contraction of renal vessels and degenerative lesions of elastic fibers lead to further elevation of the blood pressure. It is generally believed that hypertension raises renal intraglomerular pressure, which overloads the glomeruli, stimulating fibrosis and enlargement of the mesangial region, which advances to hardening of the glomeruli. In diabetic nephropathy as well, elevation in intraglomerular pressure is followed by trace albuminuria, progressing to the sclerosis of the glomeruli. Eventually, renal functions decline, resulting in chronic renal failure requiring artificial dialysis therapy. In recent years, 20% of patients with end-stage renal failure who commence artificial dialysis have diabetic nephropathy as the underlying disease. The number of patients likely to receive artificial dialysis tends to increase year after year, posing a critical problem in the medical care system. At present, it is said that there are few ideal pharmaceutical therapies for chronic renal failure, and even that blood-pressure-lowering therapy may aggravate rather than improve renal failure.

Many data of clinical and experimental studies have been reported on the relation between renal diseases and hypertension. It is now established that the kidneys are directly or indirectly involved in the onset of hypertension, and also are apt to be affected by hypertension. However, hypertension in chronic glomerulonephritis has been poorly elucidated, particularly as to causative factors, effects of hypertension on the course of nephritis, and prophylactic effects of blood pressure lowering therapy.

Currently, nephritis is considered to be a clinical picture of different diseases with different entities. In accordance with the popularization of renal biopsy, renal diseases have been reviewed, resulting in their redefinition as a wide range of diseases characterized by proteinuria (“Shibata's Internal Medicine of the Kidneys,” by Seiichi Shibata, Bunkodo, 1988). Glomerulonephritis, once regarded as a single disease, has been differentiated into glomerulonephritis, chronic pyelonephritis, IgA nephropathy, periarteritis nodosa, gout, diabetes, systemic lupus erythematosus (SLE), hepatic infarction, hereditary renal disease, amyloidosis, and Wegener's sarcoma.

Furthermore, additional types of renal disease are contemplated as being susceptible of treatment, either or both of prophylaxis and amelioration, by the compounds of formula I. These include the following: Goodpasture's syndrome, mesangiocapillary GN, Henoch-Schönlein purpura, Berger's disease (IgA nephropathy), membranous glomerulonephritis, focal segmental glomerulosclerosis, lupus nephritis, hereditary nephritis, mesangial proliferative GN, thin basement membrane disease, crescentic nephritis (also known as Rapidly Progressive GlomeruloNephritis, or RPGN), and postinfectious glomerulonephritis (PIGN).

Diabetes associated with hypertension facilitates cardiovascular impairment and/or other organ complications, greatly affecting life expectancy. Accordingly, it is important to control blood pressure within the normal range during treatment, along with the control of diabetes and the improvement or prevention of arteriosclerosis.

This invention provides a prophylactic or therapeutic drug for diabetic nephropathy or glomerular nephritis.

### SUMMARY OF THE INVENTION

We have now found that a compound of Formula I, or its pharmaceutically acceptable salts, is useful for the treatment of nephropathy or nephritis. Such a compound may be effective in the prophylaxis or treatment of diabetic nephropathy or glomerulonephritis. The subject compound or such salts are inhibitors of Syk kinase.

Namely, this invention relates to a prophylactic or therapeutic drug for diabetic nephropathy or glomerulonephritis, containing, as the active constituent, a compound or salt thereof represented by Formula I below.

A method is also disclosed for the treatment of diabetic nephropathy or glomerulonephritis in a mammal comprising the step of administering a pharmaceutically effective amount of a compound represented by Formula I below or as pharmaceutically acceptable salt thereof.

The synthesis of this compound has been disclosed in the international patent application: WO2008/033798.

### SUMMARY OF THE INVENTION

The present invention relates to a method of treating glomerulonephritis using a compound of Formula I:

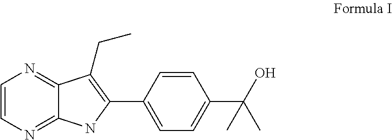

This invention is directed to a compound of Formula I, which has now been found to be active in an animal model for glomerulonephritis.

Another aspect of the present invention is a pharmaceutical composition for treating glomerulonephritis.

Another aspect of the present invention is a treatment for intraglomerular inflammation.

Yet another aspect of the present invention is a treatment for glomerulonephritis by treating a patient with a Syk inhibitor in general.

The compound of Formula I may be used as an effective oral treatment for glomerulonephritis. Furthermore we envision based on our data that Syk inhibitors in general can be useful agents for the treatment of this disorder. Our data for these conclusions is outlined below.

### BRIEF DESCRIPTION OF THE DRAWING

The above and other aspects, features, and advantages of the present invention will be better understood from the following detailed description taken in conjunction with the accompanying drawings, all of which are given by way of illustration only, and are not limitative of the present invention, in which:

FIG. 1: Percent Inhibition of total urinary protein levels (mg) in an animal model of glomerulonephritis.

### DETAILED DESCRIPTION OF THE INVENTION

Thus, in one aspect, the present invention is directed to pharmaceutical compositions comprising a compound of general Formula I, which also may be known as: 2-[4-(7-Ethyl-5H-pyrrolo [2,3-b]pyrazin-6-yl)-phenyl]-propan-2-ol.

In the present specification, the term “compound of the invention”, and equivalent expressions, are meant to embrace a compound of general formula (I) as hereinbefore described, which expression includes the ester prodrugs, the pharmaceutically acceptable salts, and the solvates, e.g. hydrates, where the context so permits. Similarly, reference to intermediates, whether or not they themselves are claimed, is meant to embrace their salts, and solvates, where the context so permits. For the sake of clarity, particular instances when the context so permits are sometimes indicated in the text, but these instances are purely illustrative and it is not intended to exclude other instances when the context so permits.

The synthesis of this compound has been disclosed in the international patent application: WO2008/033798.

The contents of each of the patent documents and other references cited herein are herein incorporated by reference in their entirety.

### List of Abbreviations

As used above, and throughout the description of the invention, the following abbreviations, unless otherwise indicated, shall be understood to have the following meanings:
ACN acetonitrile
AIBN 2,2′-azobisisobutyronitrile
bid twice daily
BOC or Boc tert-butyl carbamate
BOP benzotriazol-1-yl-oxytris (dimethylamino) phosphonium
n-Bu₃SnH tri-n-butyltin hydride
t-Bu tert-butyl
Cbz benzyl carbamate
PTC phase transfer catalyst
DAST (diethylamino) sulfur trifluoride (Et₂NSF₃)
DCC dicyclohexylcarbodiimide
DCM dichloromethane (CH₂CI₂)
DIC 1,3-diisopropylcarbodiimide
DIPEA diisopropylethylamine
DMAP 4-(N,N-dimethylamino)pyridine
DMP reagent Dess-Martin Periodinane reagent
DMF dimethylformamide
DMSO dimethylsulfoxide
EA elemental analysis
EDCI 1-ethyl-3-(3-dimethylaminopropyl) carbodiimide HCl
eq equivalent(s)
Et ethyl
Et₂O diethyl ether
EtOH ethanol
EtOAc ethyl acetate
FMOC 9-fluorenylmethoxycarbonyl
HOAt 1-hydroxy-7-azabenzotriazole
HOBT 1-hydroxybenzotriazole
HOSu N-hydroxysuccinamide
HPLC high performance liquid chromatography
LAH lithium aluminum anhydride
Me methyl
MeI methyliodide
MeOH methanol
MeOC(O) methyl chloroformate
MOMCI methoxymethylchloride
MOM methoxymethyl
MS mass spectroscopy
NaBH₄ sodium borohydride
Na₂C₄H₄O₆ sodium tartrate
NMR nuclear magnetic resonance
P Polymer bond
PO per oral administration
PyBOP benzotriazole-1-yl-oxytris-pyrrolidino-phosphonium hexafluorophosphate
TBD 1,5,7-triazabicyclo[4.4.0]-dec-5-ene
RP-HPLC reverse phase-high pressure liquid chromatography
TBSCI tert-butyldimethylsilyl chloride
TCA trichloroacetic acid
TFA trifluoroacetic acid
Tf₂O triflate anhydride
THF tetrahydrofuran
THP tetrahydropyran
TLC thin layer chromatography

### DEFINITIONS

As used above, and throughout the description of the invention, the following terms, unless otherwise indicated, shall be understood to have the following meanings.

“Acid bioisostere” means a group which has chemical and physical similarities producing broadly similar biological properties to a carboxy group (see Lipinski, Annual Reports in Medicinal Chemistry, “Bioisosterism In Drug Design” 21, 283 (1986); Yun, Hwahak Sekye, “Application of Bioisosterism To New Drug Design” 33, 576-579, (1933); Zhao, Huaxue Tongbao, “Bioisosteric Replacement And Development Of Lead Compounds In Drug Design” 34-38, (1995); Graham, Theochem, “Theoretical Studies Applied To Drug Design ab initio Electronic Distributions In Bioisosteres” 343, 105-109, (1995)). Exemplary acid bioisosteres include —C(O)—NHOH, —C(O)—CH2OH, —C(O)—CH2SH, —C(O)—NH—CN, sulfo, phosphono, alkylsulfonylcarbamoyl, tetrazolyl, arylsulfonylcarbamoyl, N-methoxycarbamoyl, heteroarylsulfonylcarbamoyl, 3-hydroxy-3-cyclobutene-1,2-dione, 3,5-dioxo-1,2,4-oxadiazolidinyl or hydroxyheteroaryl such as 3-hydroxyisoxazolyl, 3-hydroxy-1-methylpyrazolyl and the like.

“Effective amount” is means an amount of a compound/composition according to the present invention effective in producing the desired therapeutic effect.

“Hydrate” means a solvate wherein the solvent molecule {s) is/are H₂O.

“Patient” includes both human and other mammals.

“Pharmaceutically acceptable ester” refers to esters that hydrolyze in vivo and include those that break down readily in the human body to leave the parent compound or a salt thereof, Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Exemplary esters include formates, acetates, propionates, butyrates, acrylates, ethylsuccinates, and the like.

“Pharmaceutically acceptable prodrugs” as used herein refers to those prodrugs of the compounds of the present invention which are, within the scope of sound medical judgment, suitable for use in contact with the tissues of patients with undue toxicity, irritation, allergic response, and the like, commensurate with a reasonable benefit/risk ratio, and effective for their intended use of the compounds of the invention. The term “prodrug” refers to compounds that are rapidly transformed in vivo to yield the parent compound of the above formula, for example by hydrolysis in blood. Functional groups that may be rapidly transformed, by metabolic cleavage, in vivo form a class of groups reactive with the carboxyl group of the compounds of this invention. They include, but are not limited to such groups as alkanoyl (such as acetyl, propanoyl, butanoyl, and the like), unsubstituted and substituted aroyl (such as benzoyl and substituted benzoyl), alkoxycarbonyl (such as ethoxycarbonyl), trialkylsilyl (such as trimethyl- and triethysilyl), monoesters formed with dicarboxylic acids (such as succinyl), and the like. Because of the ease with which the metabolically cleavable groups of the compounds of this invention are cleaved in vivo, the compounds bearing such groups act as pro-drugs. The compounds bearing the metabolically cleavable groups have the advantage that they may exhibit improved bioavailability as a result of enhanced solubility and/or rate of absorption conferred upon the parent compound by virtue of the presence of the metabolically cleavable group. A thorough discussion is provided in Design of Prodrugs, H. Bundgaard, ed., Elsevier (1985); Methods in Enzymology; K. Widder et al, Ed., Academic Press, 42, 309-396 (1985); A Textbook of Drug Design and Development, Krogsgaard-Larsen and H. Bandaged, ed., Chapter 5; “Design and Applications of Prodrugs” 113-191 (1991); Advanced Drug Delivery Reviews, H. Bundgard, 8, 1-38, (1992); J. Pharm. Sci., 77., 285 (1988); Chem. Pharm. Bull., N. Nakeya et al, 32, 692 (1984); Pro-drugs as Novel Delivery Systems, T. Higuchi and V. Stella, 14 A.C.S. Symposium Series, and Bioreversible Carriers in Drug Design, E. B. Roche, ed., American Pharmaceutical Association and Pergamon Press, 1987, which are incorporated herein by reference.

“Pharmaceutically acceptable salts” refers to the relatively non-toxic, inorganic and organic acid addition salts, and base addition salts, of compounds of the present invention. These: salts can be prepared in situ during the final isolation and purification of the compounds. In particular, acid addition salts can be prepared by separately reacting the purified compound in its free base form with a suitable organic or inorganic acid and isolating the salt thus formed. Exemplary acid addition salts include the hydrobromide, hydrochloride, sulfate, bisulfate, phosphate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptonate, lactiobionate, sulfamates, malonates, salicylates, propionates, methylene-bis-B-hydroxynaphthoates, gentisates, isethionates, di-p-toluoyltartrates, methanesulfonates, ethanesulfonates, benzenesulfonates, p-toluenesulfonates, cyclohexylsulfamates and laurylsulfonate salts, and the like. See, for example S. M. Berge, et al., “Pharmaceutical Salts,” J. Pharm. Sci., 66, 1-19 (1977) which is incorporated herein by reference. Base addition salts can also be prepared by separately reacting the purified compound in its acid form with a suitable organic or inorganic base and isolating the salt thus formed. Base addition salts include pharmaceutically acceptable metal and amine salts. Suitable metal salts include the sodium, potassium, calcium, barium, zinc, magnesium, and aluminum salts. The sodium and potassium salts are preferred. Suitable inorganic base addition salts are prepared from metal bases which include sodium hydride, sodium hydroxide, potassium hydroxide, calcium hydroxide, aluminum hydroxide, lithium hydroxide, magnesium hydroxide, zinc hydroxide and the like. Suitable amine base addition salts are prepared from amines which have sufficient basicity to form a stable salt, and preferably include those amines which are frequently used in medicinal chemistry because of their low toxicity and acceptability for medical use. Suitable amines include ammonia, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e.g., lysine and arginine, and dicyclohexylamine, and the like.

“Solvate” means a physical association of a compound of this invention with one or more solvent molecules. This physical association includes hydrogen bonding. In certain instances the solvate will be capable of isolation, for example when one or more solvent molecules are incorporated in the crystal lattice of the crystalline solid. “Solvate” encompasses both solution-phase and isolable solvates. Exemplary solvates include hydrates, ethanolates, methanolates, and the like.

“Treating” and “Treatment” mean administration of a compound to either ameliorate a disease condition or disorder, or prevent a disease condition or disorder. Or, the slowing of the progression of the disease condition or disorder. And these also refer to reducing susceptibility to a disease condition or disorder. The terms also include but are not limited to palliative therapy that is non-curative.

### EMBODIMENTS

With reference to inventions described herein, below are particular embodiments related thereto.

A particular embodiment of the invention is a method of treating glomerulonephritis, comprising: administering to a patient in need thereof an effective amount of a compound of Formula I:

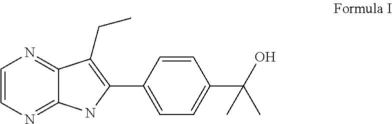

or a corresponding N-oxide, prodrug, pharmaceutically acceptable salt or solvate thereof.

Another particular embodiment of the invention is a pharmaceutical composition for treating glomerulonephritis, comprising a compound of formula I, or a corresponding N-oxide, prodrug, pharmaceutically acceptable salt or salt thereof, in combination with a pharmaceutically acceptable excipient.

Yet another embodiment of the invention is a method for the treatment of a human or non-human animal patient suffering from, or subject to, a condition that can be ameliorated by the administration of a pharmaceutically effective amount of a compound of formula I:

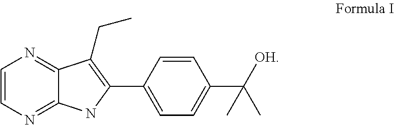

Yet another embodiment of the invention is a method of treating glomerulonephritis, comprising: administering to a patient in need thereof an effective amount of a compound which is a Syk inhibitor.

A further embodiment of the invention is a method of treating a renal disease, comprising: administering to a patient in need thereof an effective amount of a compound of Formula I:

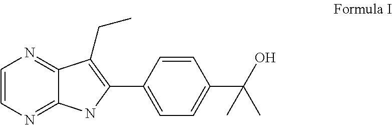

or a corresponding N-oxide, prodrug, pharmaceutically acceptable salt or solvate thereof, wherein the renal disease is selected from: glomerulonephritis, Goodpasture's syndrome, mesangiocapillary GN, post-infectious glomerulonephritis, Henoch-Schönlein purpura, Berger's disease (IgA nephropathy), membranous glomerulonephritis, focal segmental glomerulosclerosis, lupus nephritis, hereditary nephritis, mesangial proliferative GN, thin basement membrane disease, and crescentic nephritis (also known as Rapidly Progressive GlomeruloNephritis, or RPGN).

And yet another embodiment of the invention is a method of treating glomerulonephritis, comprising administering to a patient in need thereof a compound of formula I, wherein the compound is in the form of a hydrochloride salt.

The compounds of the invention optionally are supplied as salts. Those salts that are pharmaceutically acceptable are of particular interest since they are useful in administering the foregoing compounds for medical purposes. Salts that are not pharmaceutically acceptable are useful in manufacturing processes, for isolation and purification purposes, and in some instances, for use in separating stereoisomeric forms of the compounds of this invention. The latter is particularly true of amine salts prepared from optically active amines.

Where the compound of the invention contains a carboxy group, or a sufficiently acidic bioisostere, base addition salts may be formed and are simply a more convenient form of use; and in practice, use of the salt form inherently amounts to use of the free acid form.

Also, where the compound of the invention contains a basic group, or a sufficiently basic bioisostere, acid addition salts may be formed and are simply a more convenient form for use; and in practice, use of the salt form inherently amounts to use of the free base form.

Another object of the present invention is to provide a pharmaceutical composition comprising, a pharmaceutically effective amount of a compound of formula I and pharmaceutically acceptable carrier or diluent.

It is another object of the invention to provide a pharmaceutical composition which is effective, in and of itself, for utilization in a beneficial combination therapy because it includes a plurality of active ingredients which may be utilized in accordance with the invention.

The invention also provides kits or single packages combining two or more active ingredients useful in treating or preventing macular degeneration in a patient. A kit may provide (alone or in combination with a pharmaceutically acceptable diluent or carrier), the compound of formula I and the additional active ingredient (alone or in combination with diluent or carrier).

Compounds of formula I may be prepared by the application or adaptation of known methods as used heretofore or described in the literature, or by methods disclosed herein.

The amount of the compound of Formula I in any of the foregoing applications can be a pharmaceutically effective amount, a suboptimal effective amount, or combinations thereof, so long as the final combination of ingredients comprises a pharmaceutically effective amount of compounds that is effective in treating or preventing macular degeneration in a patient.

### Preparation Of Compounds Of The Invention

The starting materials and intermediates of compounds of the invention may be prepared by the application or adaptation of known methods.

Compounds of the invention may be prepared by the application or adaptation of known methods, by which is meant methods used heretofore or described in the literature, for example those described by R. C. Larock in Comprehensive Organic Transformations, VCH publishers (1989).

In particular, the compound of Formula I may be prepared as described in the international patent application WO2008/033798.

According to a further feature of the present invention, compounds of the invention may be prepared by interconversion of other compounds of the invention.

Acid addition salts are formed with the compounds of the invention in which a basic function such as an imino nitrogen, amino or mono or disubstituted group is present. A particular acid addition salt is the pharmaceutically acceptable acid addition salt, i.e., a salt whose anion is non-toxic to a patient in a pharmaceutical dose of the salt, so that the beneficial effects inherent in the free acid are not initiated by side effects ascribable to the anion. The salts chosen are chosen optimally to be compatible with the customary pharmaceutical vehicles and adapted for oral or parenteral administration. Acid addition salts of the compounds of this invention may be prepared by reaction of the free base with the appropriate acid, by the application or adaptation of known methods. For example, the acid addition salts of the compounds of this invention may be prepared either by dissolving the fee base in water or aqueous alcohol solution or other suitable solvents containing the appropriate acid and isolating the salt by evaporating the solution, or by reacting the free base and acid in an organic solvent, in which case the salt separates directly or can be obtained by concentration of the solution. Some suitable acids for use in the preparation of such salts are hydrochloric acid, hydrobromic acid, phosphoric acid, sulfuric acid, various organic carboxylic and sulfonic acids, such as acetic acid, citric acid, propionic acid, succinic acid, benzoic acid, tartaric acid, fumaric acid, mandelic acid, ascorbic acid, malic acid, methanesulfonic acid, toluenesulfonic acid, fatty acid, mandelic acid, ascorbic acid, malic acid, methanesulfonic acid, toluenesulfonic acid, and fatty acids. Other acid addition salts include adipate alginate, ascorbate, aspartate, benzenesulfonate, benzoate, cyclopentanepropionate, digluconate, dodecylsulfate, bisulfate, butyrate, lactate, laurate, lauryl sulfate, maleate, hydroiodide, 2-hydroxy-ethanesulfonate, glycerophosphate, picrate, pivalate, pamoate, pectinate, persulfate, 3-phenylpropionate, thiocyanate, 2-naphthalenesulfonate, undecanoate, nicotinate, hemisulfate, heptonate, hexanoate, camphorate, and camphorsulfonate salts, and others.

The acid addition salts of the compounds of this invention can be regenerated from the salts by the application or adaptation of known methods. For example, parent compounds of the invention can be regenerated from their acid addition salts by treatment with an alkali, e.g., aqueous sodium bicarbonate solution or aqueous ammonia solution.

Base addition salts may be formed where the compound of the invention contains a carboxy group, or a sufficiently acidic bioisostere. The bases which can be used to prepare the base addition salts include preferably those which produce, when combined with the free acid, pharmaceutically acceptable salt, i.e., salt whose cation is non-toxic to a patient in a pharmaceutical dose of the salt, so that the beneficial effects inherent in the free base are not vitiated by side effects ascribable to the cation.

Pharmaceutically acceptable salts, including those derived from alkali and alkaline earth metal salts within the scope of the invention include those derived from the following bases: sodium hydride, sodium hydroxide, potassium hydroxide, calcium hydroxide, aluminum hydroxide, lithium hydroxide, magnesium hydroxide, zinc hydroxide, ammonia, ethylenediamine, N-methyl-glucamine, lysine, arginine, ornithine, choline. N,N′-dibenzylethylenediamine, chloroprocaine, diethanolamine, procaine, N-benzylphenethylamine, diethylamine, piperazine, tris(hydroxymethyl)-aminomethane. tetramethylammonium hydroxide, and the like.

Compounds of this invention can be regenerated from their base addition salts by the application or adaptation of known methods. For example, parent compounds of the invention can be regenerated from their base addition salts by treatment with an acid, e.g., hydrochloric acid.

Compounds of the present invention may be conveniently prepared, or formed during the process of the invention, as solvates (e.g., hydrates). Hydrates of compounds of the present invention may be conveniently prepared by recrystallization from an aqueous/organic solvent mixture, using organic solvents such as dioxin, tetrahydrofuran or methanol.

### Pharmacology

Compounds according to the invention as described herein as being useful for being able to inhibit Syk kinase, and are also useful for treating glomerulonephritis and related kidney disorders. In particular, the compound of Formula I has been shown to be a Syk kinase inhibitor as described in the international patent application WO2008/033798.

A particular aspect of the invention provides for a compound according to the invention to be administered in the form of a pharmaceutical composition, though the compound may be administered alone. “Pharmaceutical composition” means a composition comprising a compound of formula 1 and at least one component selected from the group comprising pharmaceutically acceptable carriers, diluents, coatings, adjuvants, excipients, or vehicles, such as preserving agents, fillers, disintegrating agents, wetting agents, emulsifying agents, emulsion stabilizing agents, suspending agents, isotonic agents, sweetening agents, flavoring agents, perfuming agents, coloring agents, antibacterial agents, antifungal agents, other therapeutic agents, lubricating agents, adsorption delaying or promoting agents, and dispensing agents, depending on the nature of the mode of administration and dosage forms. The compositions may be presented in the form of tablets, pills, granules, powders, aqueous solutions or suspensions, injectable solutions, elixirs or syrups. Exemplary suspending agents include ethoxylated isostearyl alcohols, polyoxyethylene sorbitol and sorbitan esters, microcrystalline cellulose, aluminum metahydroxide, bentonite, agar-agar and tragacanth, or mixtures of these substances. Exemplary antibacterial and antifungal agents for the prevention of the action of microorganisms include parabens, chlorobutanol, phenol, sorbic acid, and the like. Exemplary isotonic agents include sugars, sodium chloride and the like. Exemplary adsorption delaying agents to prolong absorption include aluminum monostearate and gelatin. Exemplary adsorption promoting agents to enhance absorption include dimethyl sulfoxide and related analogs. Exemplary carriers, diluents, solvents, vehicles, solubilizing agents, emulsifiers and emulsion stabilizers, include water, chloroform, sucrose, ethanol, isopropyl alcohol, ethyl carbonate, ethyl acetate, benzyl alcohol, tetrahydrofurfuryl alcohol, benzyl benzoate, polyols, propylene glycol, 1,3-butylene glycol, glycerol, polyethylene glycols, dimethylformamide, Tween® 60, Span® 60, cetostearyl alcohol, myristyl alcohol, glyceryl mono-stearate and sodium lauryl sulfate, fatty acid esters of sorbitan, vegetable oils (such as cottonseed oil, groundnut oil, com germ oil, olive oil, castor oil and sesame oil) and injectable organic esters such as ethyl oleate, and the like, or suitable mixtures of these substances. Exemplary excipients include lactose, milk sugar, sodium citrate, calcium carbonate, dicalcium phosphate. Exemplary disintegrating agents include starch, alginic acids and certain complex silicates. Exemplary lubricants include magnesium stearate, sodium lauryl sulfate, talc, as well as high molecular weight polyethylene glycols.

Other therapeutic agents may be used in combination with a compound of the present invention. Therapeutic agents used in combination with a compound of the present invention may be administered separately, simultaneously or sequentially. The choice of material in the pharmaceutical composition other than the compound of formula 1 is generally determined in accordance with the chemical properties of the active compound such as solubility, the particular mode of administration and the provisions to be observed in pharmaceutical practice. For example, excipients such as lactose, sodium citrate, calcium carbonate, dicalcium phosphate and disintegrating agents such as starch, alginic acids and certain complex silicates combined with lubricants such as magnesium stearate, sodium lauryl sulfate and talc may be used for preparing tablets.

The pharmaceutical compositions may be presented in assorted forms such as tablets, pills, granules, powders, aqueous solutions or suspensions, injectable solutions, elixirs or syrups.

“Liquid dosage form” means the dose of the active compound to be administered to the patient is in liquid form, for, example, pharmaceutically acceptable emulsions, solutions, suspensions, syrups and elixirs. In addition to the active compounds, the liquid dosage forms may contain inert diluents commonly used in the art, such solvents, solubilizing agents and emulsifiers.

Solid compositions may also be employed as fillers in soft and hard-filled gelatin capsules using such excipients as lactose or milk sugar as well as high molecular weight polyethylene glycols, and the like.

When aqueous suspensions are used they can contain emulsifying agents or agents which facilitate suspension.

The oily phase of the emulsion pharmaceutical composition may be constituted from known ingredients in a known manner. While the phase may comprise merely an emulsifier (otherwise known as an emulgent), it desirably comprises a mixture of at least one emulsifier with a fat or an oil or with both a fat and an oil. In a particular embodiment, a hydrophilic emulsifier is included together with a lipophilic emulsifier that acts as a stabilizer. Together, the emulsifier(s) with or without stabilizer(s) make up the emulsifying wax, and the way together with the oil and fat make up the emulsifying ointment base which forms the oily dispersed phase of the cream formulations.

If desired, the aqueous phase of the cream base may include, for example, a least 30% w/w of a polyhydric alcohol, i.e. an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound that enhances absorption or penetration of the active ingredient through the skin or other affected areas.

The choice of suitable oils or fats for a formulation is based on achieving the desired properties. Thus the cream should preferably be a non-greasy, non-staining and washable product with suitable consistency to avoid leakage from tubes or other containers. Straight or branched chain, mono- or dibasic alkyl esters such as di-isopropyl myristate, decyl oleate, isopropyl palmitate, butyl stearate, 2-ethylhexyl palmitate or a blend of branched chain esters known as Crodamol CAP may be used. These may be used alone or in combination depending on the properties required. Alternatively, high melting point lipids such as white soft paraffin and/or liquid paraffin or other mineral oils can be used.

In practice, a compound/pharmaceutical compositions of the present invention may be administered in a suitable formulation to humans and animals by topical or systemic administration, including oral, inhalational, rectal, nasal, buccal, sublingual, vaginal, colonic, parenteral (including subcutaneous, intramuscular, intravenous, intradermal, intrathecal and epidural), intracisternal and intraperitoneal. It will be appreciated that the preferred route may vary with for example the condition of the recipient.

“Pharmaceutically acceptable dosage forms” refers to dosage forms of the compound of the invention, and includes, for example, tablets, dragées, powders, elixirs, syrups, liquid preparations, including suspensions, sprays, inhalants tablets, lozenges, emulsions, solutions, granules, capsules and suppositories, as well as liquid preparations for injections, including liposome preparations. Techniques and formulations generally may be found in Remington's Pharmaceutical Sciences, Mack Publishing Co., Easton, Pa., latest edition.

“Formulations suitable for oral administration” may be presented as discrete units such as capsules, cachets or tablets each containing a predetermined amount of the active ingredient; as a powder or granules; as solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tables may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered compounds moistened with an inert liquid diluent. The tablets may optionally be coated or scored and may be formulated so as to provide slow or controlled release of the active ingredient therein.

Solid compositions for rectal administration include suppositories formulated in accordance with known methods and containing at least one compound of the invention.

If desired, and for more effective distribution, the compounds can be microencapsulated in, or attached to, a slow release or targeted delivery systems such as a biocompatible, biodegradable polymer matrices (e.g., poly(d,l-lactide co-glycolide)), liposomes, and microspheres and subcutaneously or intramuscularly injected by a technique called subcutaneous or intramuscular depot to provide continuous slow release of the compound(s) for a period of 2 weeks or longer. The compounds may be sterilized, for example, by filtration through a bacteria retaining filter, or by incorporating sterilizing agents in the form of sterile solid compositions which can be dissolved in sterile water, or some other sterile injectable medium immediately before use.

“Formulations suitable for nasal or inhalational administration” means formulations which are in a form suitable to be administered nasally or by inhalation to a patient. The formulation may contain a carrier, in a powder form, having a particle size for example in the range 1 to 500 microns (including particle sizes in a range between 20 and 500 microns in increments of 5 microns such as 30 microns, 35 microns, etc.). Suitable formulations wherein the carrier is a liquid, for administration as for example a nasal spray or as nasal drops, include aqueous or oily solutions of the active ingredient. Formulations suitable for aerosol administration may be prepared according to conventional methods and may be delivered with other therapeutic agents. Inhalational therapy is readily administered by metered dose inhalers.

“Formulations suitable for oral administration” means formulations which are in a form suitable to be administered orally to a patient. The formulations may be presented as discrete units such as capsules, cachets or tablets each containing a predetermined amount of the active ingredient; as a powder or granules; as solution or a suspension in an aqueous liquid or a non-aqueous liquid; or as an oil-in-water liquid emulsion or a water-in-oil liquid emulsion. The active ingredient may also be presented as a bolus, electuary or paste.

“Formulations suitable for parenteral administration” means formulations that are in a form suitable to be administered parenterally to a patient. The formulations are sterile and include emulsions, suspensions, aqueous and non-aqueous injection solutions, which may contain suspending agents and thickening agents and anti-oxidants, buffers, bacteriostats and solutes which render the formulation isotonic, and have a suitably adjusted pH, with the blood of the intended recipient.

“Formulations suitable for rectal or vaginal administrations” means formulations that are in a form suitable to be administered rectally or vaginally to a patient. Suppositories are a particular form for such formulations that can be prepared by mixing the compounds of this invention with suitable non-irritating excipients or carriers such as cocoa butter, polyethylene glycol or a suppository wax, which are solid at ordinary temperatures but liquid at body temperature and therefore, melt in the rectum or vaginal cavity and release the active component.

“Formulations suitable for systemic administration” means formulations that are in a form 20 suitable to be administered systemically to a patient. The formulation is preferably administered by injection, including transmuscular, intravenous, intraperitoneal, and subcutaneous. For injection, the compounds of the invention are formulated in liquid solutions, in particular in physiologically compatible buffers such as Hank's solution or Ringer's solution. In addition, the compounds may be formulated in solid form and redissolved or suspended immediately prior to use. Lyophilized forms are also included. Systematic administration also can be by transmucosal or transdermal means, or the compounds can be administered orally. For transmucosal or transdermal administration, penetrants appropriate to the barrier to be permeated are used in the formulation. Such penetrants are generally known in the art, and include, for example, bile salts and fusidic acid derivatives for transmucosal administration. In addition, detergents may be used to facilitate permeation. Transmucosal administration may be through use of nasal sprays, for example, or suppositories. For oral administration, the compounds are formulated into conventional oral administration forms such as capsules, tablets, and tonics.

“Formulations suitable for topical administration” means formulations that are in a form suitable to be administered topically to a patient. The formulation may be presented as a topical ointment, salves, powders, sprays and inhalants, gels (water or alcohol based), creams, as is generally known in the art, or incorporated into a matrix base for application in a patch, which would allow a controlled release of compound through the transdermal barrier. When formulated in an ointment, the active ingredients may be employed with either a paraffinic or a water-miscible ointment base. Alternatively, the active ingredients may be formulated in a cream with an oil-in-water cream base. Formulations suitable for topical administration in the eye include eye drops wherein the active ingredient is dissolved or suspended in a suitable carrier, especially an aqueous solvent for the active ingredient. Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

“Solid dosage form” means the dosage form of the compound of the invention is solid form, for example capsules, tablets, pills, powders, dragées or granules. In such solid dosage forms, the compound of the invention is admixed with at least one inert customary excipient (or carrier) such as sodium citrate or dicalcium phosphate or (a) fillers or extenders, as for example, starches, lactose, sucrose, glucose, mannitol and silicic acid, (b) binders, as for example, carboxymethylcellulose, alginates, gelatin, polyvinylpyrrolidone, sucrose and acacia, (c) humectants, as for example, glycerol, (d) disintegrating agents, as for example, agar-agar, calcium carbonate, potato or tapioca starch, alginic acid, certain complex silicates and sodium carbonate, (e) solution retarders, as for example paraffin, (f) absorption accelerators, as for example, quaternary ammonium compounds, (g) wetting agents, as for example, cetyl alcohol and glycerol monostearate, (h) adsorbents, as for example, kaolin and bentonite, (i) lubricants, as for example, talc, calcium stearate, magnesium stearate, solid polyethylene glycols, sodium lauryl sulfate, (j) opacifying agents, (k) buffering agents, and agents which release the compound(s) of the invention in a certain part of the intestinal tract in a delayed manner.

Actual dosage levels of active ingredient(s) in the compositions of the invention may be varied so as to obtain an amount of active ingredient(s) that is (are) effective to obtain a desired therapeutic response for a particular composition and method of administration for a patient. A selected dosage level for any particular patient therefore depends upon a variety of factors including the desired therapeutic effect, on the route of administration, on the desired duration of treatment, the etiology and severity of the disease, the patient's condition, weight, sex, diet and age, the type and potency of each active ingredient, rates of absorption, metabolism and/or excretion and other factors.

Total daily dose of the compounds of this invention administered to a patient in single or divided doses may be in amounts, for example, of from about 0.001 to about 100 mg/kg body weight daily and preferably 0.01 to 10 mg/kg/day. For example, in an adult, the doses are generally from about 0.01 to about 100, preferably about 0.01 to about 10, mg/kg body weight per day by inhalation, from about 0.01 to about 100, preferably 0.1 to 70, more especially 0.5 to 10, mg/kg body weight per day by oral administration, and from about 0.01 to about 50, preferably 0.01 to 10, mg/kg body weight per day by intravenous administration. The percentage of active ingredient in a composition may be varied, though it should constitute a proportion such that a suitable dosage shall be obtained. Dosage unit compositions may contain such amounts of such submultiples thereof as may be used to make up the daily dose. Obviously, several unit dosage forms may be administered at about the same time. A dosage may be administered as frequently as necessary in order to obtain the desired therapeutic effect. Some patients may respond rapidly to a higher or lower dose and may find much weaker maintenance doses adequate. For other patients, it may be necessary to have long-term treatments at the rate of 1 to 4 doses per day, in accordance with the physiological requirements of each particular patient. It goes without saying that, for other patients, it will be necessary to prescribe not more than one or two doses per day.

The formulations can be prepared in unit dosage form by any of the methods well known in the art of pharmacy. Such methods include the step of bringing into association the active ingredient with the carrier that constitutes one or more accessory ingredients. In general the formulations are prepared by uniformly and intimately bringing into association the active ingredient with liquid carriers or finely divided solid carriers or both, and then, if necessary, shaping the product.

The formulations may be presented in unit-dose or multi-dose containers, for example sealed ampoules and vials with elastomeric stoppers, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

Compounds within the scope of the present invention exhibit marked pharmacological activities according to tests described in the literature and below, which tests results are believed to correlate to pharmacological activity in humans and other mammals.

The chemical reactions described in the references cited above are generally disclosed in terms of their broadest application to the preparation of the compounds of this invention. Occasionally, the reactions may not be applicable as described to each compound included within the scope of compounds disclosed herein. The compounds for which this occurs will be readily recognized by those skilled in the art. In all such cases, either the reactions can be successfully performed by conventional modifications known to those skilled in the art, e.g., by appropriate protection of interfering groups, by changing to alternative conventional reagents, by routine modification of reaction conditions, and the like, or other reactions disclosed herein or otherwise conventional will be applicable to the preparation of the corresponding compounds of this invention. In all preparative methods, all starting materials are known or readily preparable from known starting materials.

The regimen for treating a patient suffering from glomerulonephritis with the compound and/or compositions of the present invention is selected in accordance with a variety of factors, including the age, weight, sex, diet, and medical condition of the patient, the severity of the infection, the route of administration, pharmacological considerations such as the activity, efficacy, pharmacokinetic, and toxicology profiles of the particular compounds employed, and whether a drug delivery system is utilized. Administration of the drug combinations disclosed herein should generally be continued over a period until acceptable, indicating that has been controlled or eradicated. Patients undergoing treatment with the drug combinations disclosed herein can be routinely monitored by conventional methods of measuring kidney function to determine the effectiveness of therapy. Continuous analysis of the data obtained by these methods permits modification of the treatment regimen during therapy so that optimal amounts of each component in the combination are administered, and so that the duration of treatment can be determined as well. Thus, the treatment regimen/dosing schedule can be rationally modified over the course of therapy so that the lowest amounts of each of the compounds used in combination which together exhibit satisfactory effectiveness are administered, and so that administration of such compounds in combination is continued only so long as is necessary to successfully treat the kidney disorder.

In the present invention encompasses the use of combinations of anti-VEGF inhibitors and compounds having Syk activity as described above to treat or prevent glomerulonephritis where one or more of these compounds is present in a pharmaceutically effective amount, and the other(s) is(are) present in a subclinical pharmaceutically effective or an effective amount(s) owing to their additive or synergistic effects. As used herein, the term “additive effect” describes the combined effect of two (or more) pharmaceutically active agents that is equal to the sum of the effect of each agent given alone. A synergistic effect is one in which the combined effect of two (or more) pharmaceutically active agents is greater than the sum of the effect of each agent given alone.

### In-Vivo Efficacy of the Compound of Formula I

Study Objective: Glomerulonephritis is a complex disease involving cytokines, cell infiltration, cell proliferation, and rapid deterioration of renal function. This animal model will provide a relatively short term and quantitative measurement of glomerulonephritis. This study was designed to determine if a compound of Formula I will reduce glomerulonephritis induced by a 30 microgram intravenous injection of clone a84 (IgG2a). This monoclonal antibody induces disease by binding to a glomerular basement membrane protein. Disease progression is monitored in two ways in this model. First, the levels of protein in excreted urine are assessed. This readout serves as a rapid marker of renal diseased. The second mechanism by which disease is monitored is by examining the substructure of the kidney via histological examination.

### Results

We observed that the compound of Formula I inhibited disease in this model as monitored by urinary protein levels in a dose dependent manner. 3.0, 10, and 30 mg/kg of the compound reduced urinary protein levels relative to controls by 25, 53 and 85% respectively. At 30 mg/kg the compound of Formula I was significantly better than the positive control (Enbrel®-an anti-TNF or tumor necrosis factor compound).

### Experimental Methodology:

### Full Study Report Embedded

### 1. General Information

Compounds: the compound of Formula I as hydrochloride salt

Aim of Study: Glomerulonephritis is a complex disease involving cytokines, cell infiltration, cell proliferation, and rapid deterioration of renal function. This animal model provided a relatively short term and quantitative measurement of glomerulonephritis. This study was designed to determine if the compound of Formula I as hydrochloride salt will reduce glomerulonephritis induced by a 30 microgram intravenous injection of clone a84 (IgG2a).

### 2. Experimental Procedure

Animals:

Species, Strain, Supplier: Rat, WKY, Charles River

Sex: Male

Date of Birth: Nov. 26, 2007

Receiving Date: Feb. 5, 2008

Weight and/or Age of Animals at the Start of Treatment: At study initiation, animals were approximately 11-12 weeks old.

Number of Animals per Group: There were 3, 4, or 8 animals per group.

Animal Housing Conditions: Animals were housed under conditions outlined in the NIH Guide for the Care and Use of Laboratory Animals in compliance with the USDA Animal Welfare Act in a fully accredited AALAC facility.

Food: Purina 5001 rodent diet and filtered water were available ad libitum.

Randomization and Identification: Subjects were selected from a healthy pool of available animals and were identified by indelible tail markings. Cage cards indicated dose groups.

Conditioning of Animals: Rats were acclimated to the facility for a minimum of 5 days prior to study initiation. The animals were not fasted prior to study initiation or at any time during the study.

Clone a84 (IgG2a) challenge:

Name, Catalog #, Lot #, and Supplier: Nephrigenic Monoclonal Antibody a84, Catalog #70215, Lot #060775, Chondrex

Dose: 30 ug/animal

Vehicle: 0.9% Sodium Chloride Injectable

Route of Administration: i.v.

Volume Administered: 0.1 ml

Duration and Frequency of Treatment: Animals in groups B through F and H through L received a 30 ug iv injection of a84 antibody on day 0 approximately 1 hour post dose.

Treatment of Controls: Animals in groups A and G received a 0.1 ml iv injection of 0.9% Sodium Chloride.

Tested Compound:

The compound of Formula I as hydrochloride salt

Doses: 3.0, 10, and 30 mg/kg

Vehicle: 0.5% Methyl Cellulose containing 0.2% Tween 80

Route of Administration: Oral gavage

Volume of Administered: 10 ml/kg

Duration and Frequency of Treatment: Each animal in groups C, D, and E received an A.M. and P.M. dose approximately 8 hours apart from day 0 through day 6. Each animal in groups I, J, and K received an A.M. and P.M. dose approximately 8 hours apart from day 0 through day 3. Dosing was initiated on day 0 approximately 1 hour prior to a84 antibody injection.

Treatment of Controls: Vehicle (0.5% methyl cellulose containing 0.2% Tween 80) 10 ml/kg. Each animal in groups A and B received an A.M. and P.M. dose approximately 8 hours apart from day 0 through day 6. Each animal in groups G and H received an A.M. and P.M. dose approximately 8 hours apart from day 0 through day 3. Dosing was initiated on day 0 approximately 1 hour prior to a84 antibody injection.

Reference Compound:

Compound and Lot #: Enbrel (etanercept), Lot D092246

Supplier: Amgen/Wyeth

Dose: 5.0 mg/kg

Vehicle: 0.9% Sodium Chloride Inj., Catalog #0409-4888-50, Lot #44-199-DK

Route of Administration: intraperitoneal (ip)

Volume of Administered: 10 ml/kg

Duration and Frequency of Treatment: Each animal in groups F and L received an A.M. dose on day 0 approximately 1 hour prior to a84 antibody injection and a PM dose on day 3.

Protein Determination:

Kit: Rat Urinary Protein Assay Kit

Supplier: Chondrex

Catalog Number: 9040

Studied Parameters/Examinations/Samples:

Study Parameters Rat urinary proteins was measured over approximately a 16 hour period from day 6 to day 7. The kidneys were harvested for histological evaluation and for possible mRNA isolation.

Evaluation Method: Rat urinary protein levels was determined by the kit described in section 2.3.

Equipment Used: 1) Mettler PM Scale 2) Mettler AE 160 Analytical Scale 3) Anesthesia Machine by Cyprane 4) Beckman GS-15R Centrifuge 5) Thermomax Microplate Reader 6) Retsch MM301 Mixer Mill

Chronology of Examinations and/or Sampling: 1) Rats were placed into six groups of eight, six groups of 4, and one group of 3 based on compound and dose administered: Group A Vehicle (0.5% methyl cellulose containing 0.2% tween 80)+IV saline injection n=8, Group B Vehicle (0.5% methyl cellulose containing 0.2% tween 80)+IV a84 antibody injection n=8, Group C Compound of Formula I as hydrochloride salt 3.0 mg/kg+IV a84 antibody injection n=8, Group D SAR397769A 10 mg/kg+IV a84 antibody injection n=8, Group E Compound of Formula I as hydrochloride salt 30 mg/kg+IV a84 antibody injection n=8, Group F Enbrel 5.0 mg/kg+IV a84 antibody injection, Group G Vehicle (0.5% methyl cellulose containing 0.2% tween 80)+IV saline injection n=4, Group H Vehicle (0.5% methyl cellulose containing 0.2% tween 80)+IV a84 antibody injection n=4, Group I Compound of Formula I as hydrochloride salt 3.0 mg/kg+IV a84 antibody injection n=4, Group J SAR397769A 10 mg/kg+IV a84 antibody injection n=4, Group K Compound of Formula I as hydrochloride salt 30 mg/kg+IV a84 antibody injection n=4, Group L Enbrel 5.0 mg/kg+IV a84 antibody injection n=4, and Group M Naïve n=3. 2) On day 0, rats were dosed one hour prior to a84 antibody injection. Animals in groups A through E, and G through K were dosed orally. Dosing continued twice daily in groups A through E from day 0 to day 6 with approximately 8 hours between doses. Dosing continued twice daily in groups G through K from day 0 to day 3 with approximately 8 hours between doses. Animals in groups F and L were dosed one hour prior to a84 antibody injection. These animals received an intraperitoneal dose. These animals were dosed again on day 3 in the PM. 3) On day 0, rats received an i.v. injection of 30 ug of a84 antibody using a 26G, ⅜″ needle. 4) The 3 naïve rats in group M were sacrificed and the kidneys were collected at some point during the study. 5) Rats were placed in metabolic cages from day 6-7 for approximately 16 hours for urine collection. Urine was frozen at −40° C. until protein analysis was performed. 6) Animals in groups G through L were sacrificed on day 4. Animals in groups A through F were sacrificed on day 7. 7) Approximately 2-3 ml of blood may be collected from each rat. 8) If collected, the whole blood was spun at 4000 rpm for 5 minutes. 9) The serum was then transferred to uniquely identified tubes and stored at minus 40° C. 10) Both kidneys were collected. The histological parameters of the kidneys were evaluated.

### 3. Expression of Results and Statistical Analysis

Results were expressed as urine protein mg/ml mean+/−standard error and mg/16 hours mean+/−standard error compared to vehicle animals. Data was exported to Passerelle V5 for statistical analysis in EverStat V5 (SAS System release 8.2 for SUN 4, SAS Institute Inc., SAS Campus Drive, Cary, N.C. 27513, USA). P<0.05 was accepted as statistically significant when compared to vehicle animals.

### Modifications to the Study Plan

1) Groups I, J, and L were eliminated from the study during a84 antibody injection due to the lack of antibody.
2) One animal was eliminated from group F following the AM dose on day 0 due to the lack of a84 antibody.

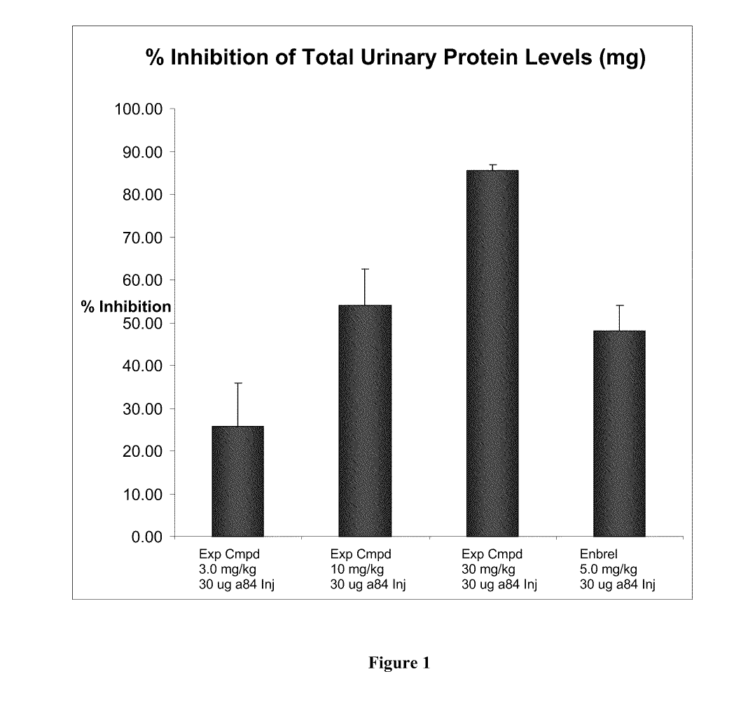

“Exp Cmpd” in Table 1 is the Compound of Formula I as hydrochloride salt. The results of the study above are shown in Table 1 in tabular form, and in FIG. 1 in graphical form. Treatment with this compound reduced the amount of total urinary protein in the experimental animal model. It would be expected from this discovery that this compound would be effective in treating glomerulonephritis in human subjects or patients.

TABLE 1
% Inhibition of Total Urinary Protein Levels (mg)

| | 30 ug a84 | 30 ug a84 | 30 ug a84 | 30 ug a84 | 30 ug a84 |
|---|---|---|---|---|---|
| | Antibody | Antibody | Antibody | Antibody | Antibody |
| | Vehicle | Exp Cmpd | Exp Cmpd | Exp Cmpd | Enbrel |
| Animal | 10 ml/kg | 3.0 mg/kg | 10 mg/kg | 30 mg/kg | 5.0 mg/kg |
| 1 | | 65.58 | 35.61 | 81.26 | 63.92 |
| 2 | | −3.63 | 11.63 | 92.32 | 22.67 |
| 3 | | −0.60 | 46.17 | 84.66 | 50.26 |
| 4 | | 9.50 | 64.93 | 85.65 | 33.67 |
| 5 | | −5.97 | 71.77 | 79.73 | 61.10 |
| 6 | | 54.11 | 76.96 | 86.92 | 46.54 |
| 7 | | 43.80 | 81.22 | 88.28 | 58.91 |
| 8 | | 42.33 | 43.56 | 84.82 | |
| Mean | | 25.64 | 53.98 | 85.46 | 48.15 |
| Stdev | | 28.83 | 23.93 | 3.94 | 15.27 |
| SEM | | 10.19 | 8.46 | 1.39 | 5.77 |

The present invention may be embodied in other specific forms without departing from the spirit or essential attributes thereof.

What is claimed is:

1. A method of treating glomerulonephritis, comprising administering to a patient in need thereof an effective amount of a compound of Formula I: 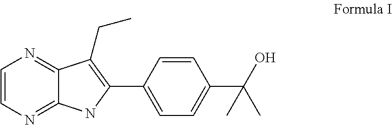
or a corresponding N-oxide, prodrug, pharmaceutically acceptable salt or solvate thereof.

2. A pharmaceutical composition for treating glomerulonephritis, comprising a compound of formula I, or a corresponding N-oxide, prodrug, pharmaceutically acceptable salt or solvate thereof, in combination with a pharmaceutically acceptable excipient.

3. A method for the treatment of a human or non-human animal patient suffering from, or subject to, a condition that can be ameliorated by the administration of a pharmaceutically effective amount of a compound of formula I: 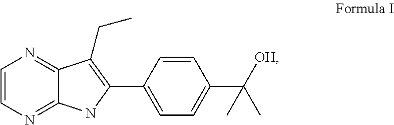
comprising administering an effective amount of said compound to a patient in need thereof.

4. A method of treating glomerulonephritis, comprising administering to a patient in need thereof an effective amount of a compound which is a Syk inhibitor.

5. A method of treating a renal disease, comprising administering to a patient in need thereof an effective amount of a compound of Formula I: 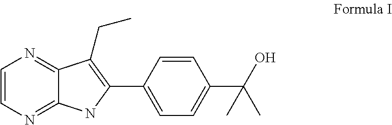
or a corresponding N-oxide, prodrug, pharmaceutically acceptable salt or solvate thereof, wherein the renal disease is selected from: glomerulonephritis, Goodpasture's syndrome, mesangiocapillary glomerulonephritis, post-infectious glomerulonephritis, Henoch-Schönlein purpura, Berger's disease, membranous glomerulonephritis, focal segmental glomerulosclerosis, lupus nephritis, hereditary nephritis, mesangial proliferative glomerulonephritis, thin basement membrane disease, crescentic nephritis, and rapidly progressive glomerulonephritis.

6. The method according to claim 1, wherein the compound is in the form of a hydrochloride salt.
